(19) United States
(12) Patent Application Publication
(10) Pub. No.: US 2026/0345572 A1
(43) Pub. Date: Nov. 5, 2026

(54) PHARMACEUTICAL FORMULATIONS WITH ACTIVITY INHIBITING HISTONE METHYLTRANSFERASES FOR THE TREATMENT OF NEOPLASTIC DISEASES

(71) Applicant: Ente Ospedaliero Specializzato in Gastroenterologia Istituto Nazionale di Ricovero e Cura a Caratter, Castellana Grotte (IT)

(72) Inventors: Alberto Del Rio, Castellana Grotte (IT); Cristiano Simone, Castellana Grotte (IT)

(73) Assignee: Ente Ospedaliero Specializzato in Gastroenterologia Istituto Nazionale di Ricovero e Cura a Caratter, Castellana Grotte (IT)

(21) Appl. No.: 19/120,447

(22) PCT Filed: Oct. 9, 2023
(86) PCT No.: PCT/IB2023/060121

§ 371 Date: Apr. 11, 2025

(30) Foreign Application Priority Data
Oct. 13, 2022 (IT) 102022000021138

(51) Int. Cl. A61K 31/454; A61K 31/282; A61K 31/337; A61K 31/4745; A61K 31/502; A61K 31/513; A61K 31/55; A61K 31/704; A61P 35/00

(52) U.S. Cl. CPC A61K 31/454; A61K 31/282; A61K 31/337; A61K 31/4745; A61K 31/502; A61K 31/513; A61K 31/55; A61K 31/704; A61P 35/00

(57) ABSTRACT

The present invention relates to novel pharmaceutical formulations given by the association of an inhibitor of histone methyltransferase activity and chemotherapeutic drugs for use in the treatment of cancer, in particular of colon cancer, breast cancer, pancreatic cancer and gastric cancer.

The present invention relates to novel pharmaceutical formulations given by the association of an inhibitor of histone methyltransferase activity and chemotherapeutic drugs for use in the treatment of cancer, in particular of colon cancer, breast cancer, pancreatic cancer and gastric cancer.

### STATE OF THE ART

It is known from the scientific literature that covalent histone modifications are involved in chromatin structure regulation and play crucial roles of the development and in the pathogenesis of several diseases. In particular, it has been demonstrated that, among covalent modifications, methylation, which is one of the major forms of histone modification, and in particular lysine methylation, performs important functions in several physiological and pathological biologic processes. For instance, it is involved in the regulation of chromatin condensation state and/or opening during embryonic development, cancerogenesis, X-chromosome inactivation, by regulation of gene transcription activity. Histone methyltransferases, which act in the abovementioned methylation process, are S-adenosyl-methionine-dependent enzymes, since they use S-adenosyl-methionine (SAM) as a methyl donor to methylate histone tails, and perform specific roles, e.g., in metabolism, cancer, neurodegeneration and inflammation.

Owing to their wide involvement in the physiopathology of highly prevalent diseases, at the present moment, in order to single out the causes or possible remedies and treatments of the latter, said histone methyltransferases represent an interesting therapeutic target. Said enzymes are able to methylate lysine or arginine residues present in chromatin histone tails. On the basis of the amino acid residue on which they act, histone methyltransferases have been subdivided into two classes: (protein) lysine methyltransferases (PKMTs) and (protein) arginine methyltransferases (PRMTs), which are encoded by 51 and 48 genes, respectively. Some studies have demonstrated that at least three PKMTs, e.g., proteins SMYD3, EZH2 and SUV39H1, are overexpressed in colorectal tumor cells and tissues; their overexpression is correlated with variations in the methylation pattern of lysine residues present in the target histones of said enzymes. In the lysine methyltransferase group (PKMT) the SMYD protein family is present, an example of which is referred to as SMYD3.

SMYD3 is an example of protein that is highly expressed in various tumor forms, among which breast, colorectal and liver carcinomas. For instance, colorectal cancer (or CRC) is the third more commonly diagnosed worldwide, and therefore has a very negative impact due to high patient mortality. Recent studies have demonstrated the oncogenic role of histone methyltransferase proteins or of SMYD family proteins, or in particular of SMYD3, i.e. their function of stimulating cancer cell proliferation, adhesion, and migration, as well as their role in muscular and inflammatory diseases.

### SUMMARY OF THE INVENTION

The Authors of the present invention have surprisingly discovered that from the association of an inhibitor of histone methyltransferase SMYD3 activity with some pharmaceutically active agents selected from one or more known chemotherapeutic drugs or PARP inhibitor drugs, an anti-tumor activity with a synergistic effect of the two (ingredients) is obtained, in particular against colon, breast, pancreatic, gastric cancer, allowing to minimize or reduce the dose of chemotherapeutic drugs (compounds) and/or PARP inhibitors, or anyhow improve patients' compliance.

The data obtained by the Inventors demonstrate, following the use on tumor cells and on the AOM/DSS murine model for the study of colorectal cancer of the association described and claimed herein, a surprising chemosensitization for some tumors and the possibility to use lower dosages of chemotherapeutic drug or PARP inhibitor, compared to those commonly used in the literature.

The inhibitor of histone methyltransferase SMYD3 activity selected by the Authors is the compound also known as EM127, having CAS Registry Number 1886879-71-5,

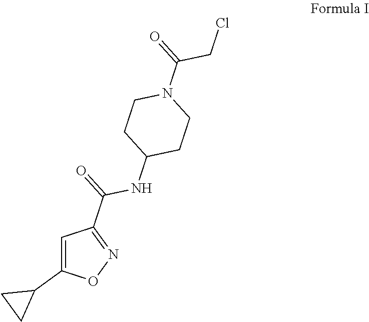 [SMILES: O=C(NC1CCN(C(=O)CCl)CC1)c1cc(C2CC2)on1]

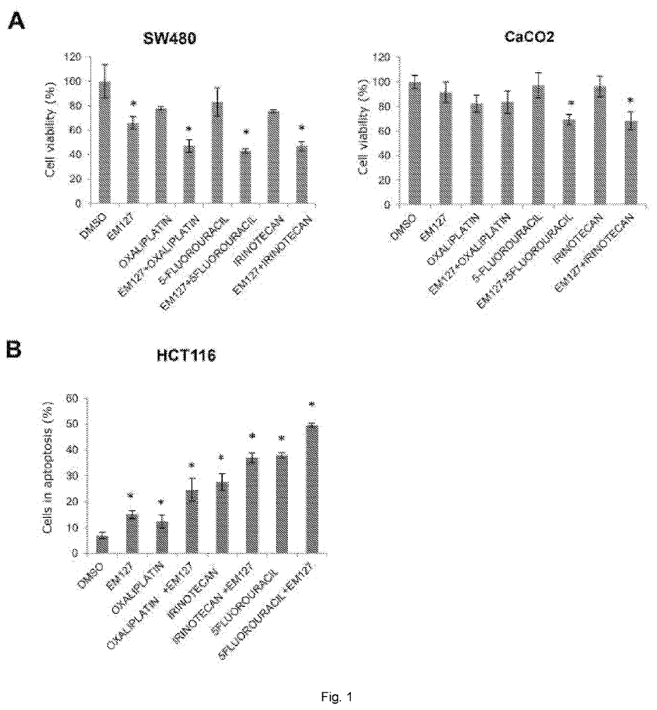

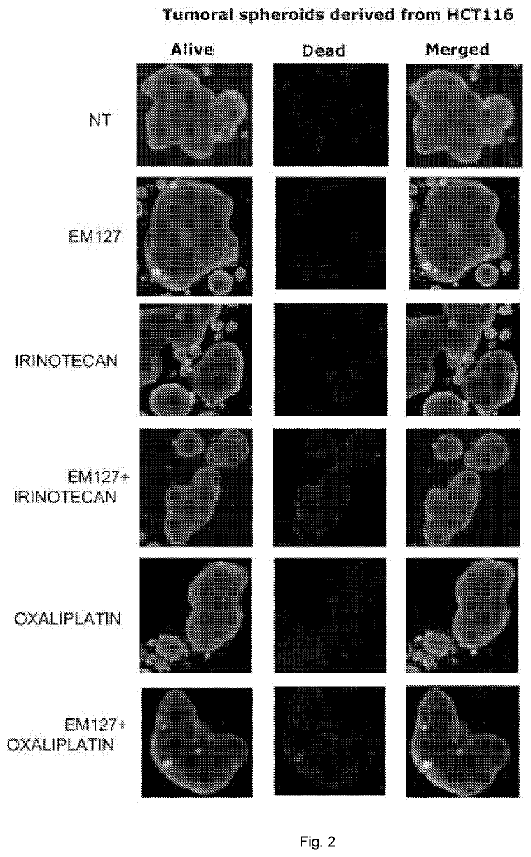

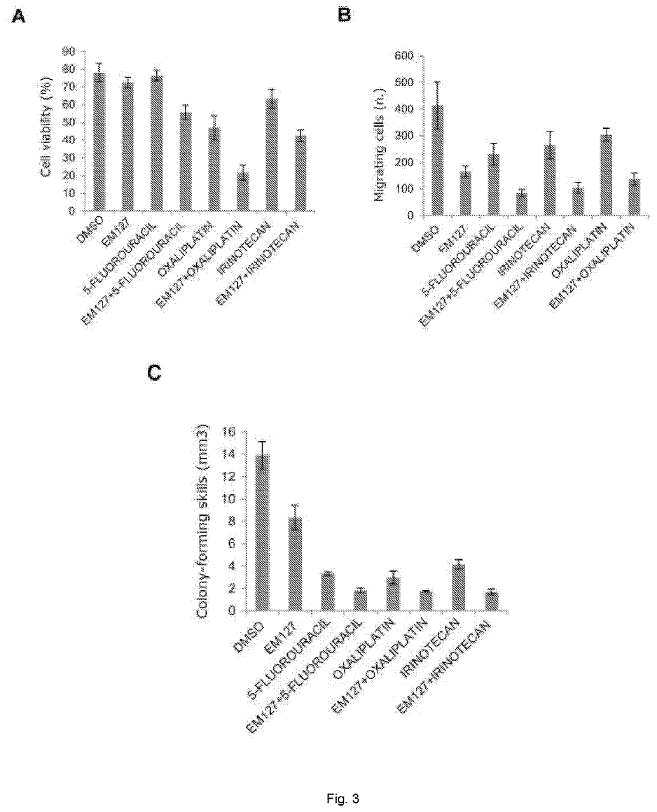

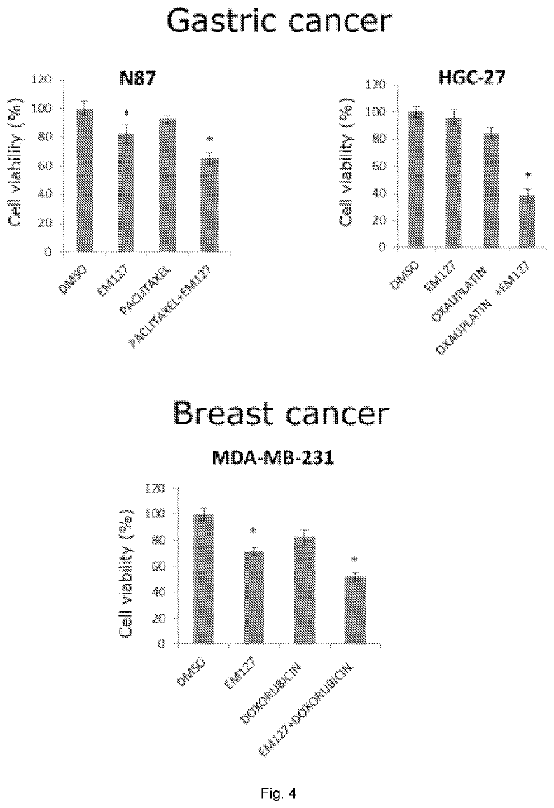

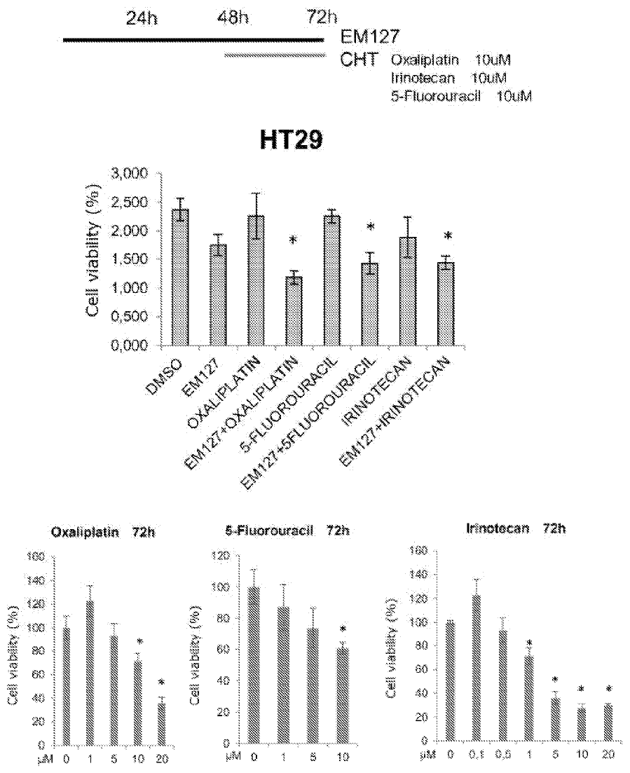

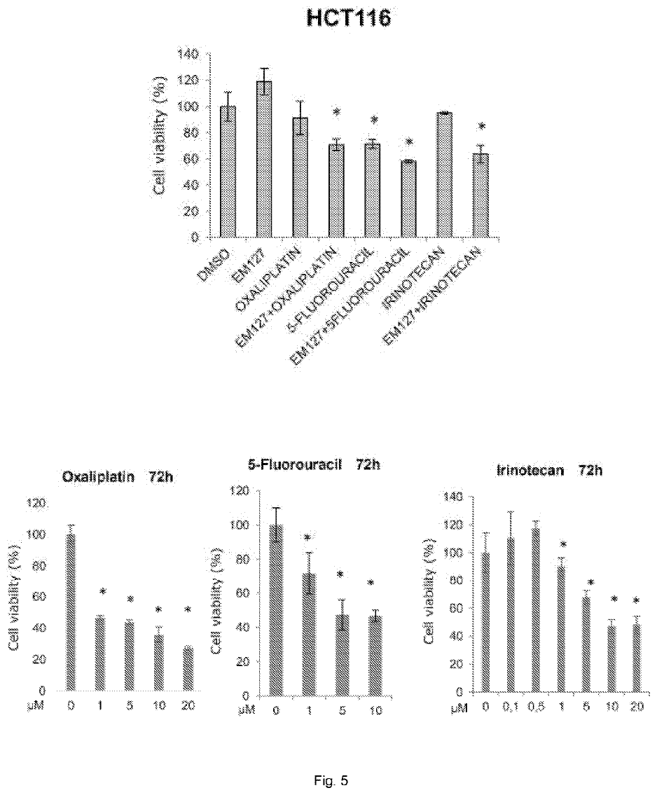

which, in association with selected chemotherapeutic compounds such as Irinotecan, Oxaliplatin, 5-Fluorouracil, Doxorubicin, Paclitaxel, or PARP inhibitors such as Olaparib, Rucaparib, showed a synergistic anti-tumor activity as demonstrated in FIGS. 1-10. In particular, the Authors of the invention demonstrated the surprising sensitization for some tumors obtainable with the association EM127 and chemotherapeutic compound or PARP inhibitor, allowing the reduction of dosages of chemotherapeutic compounds thanks to the unexpected resulting synergistic effect. In particular, it can be seen in FIG. 1 how colorectal cancer cell lines show the operational synergy between EM127 and chemotherapeutic compounds, in particular in HCT116 cell line it is observed how the combined treatment increases the number of cells undergoing programmed death (apoptosis). In FIG. 2, this synergy can be demonstrated by analyzing the effects of the combined treatment in 3D cell models obtained from HCT116 cell line (HCT116-derived tumor spheroids), which exhibit a good stem cell component, a population able to organize into well-defined spheroidal structures called tumorspheres and accountable for resistance to therapies. In FIG. 3 it is apparent how the EM127 combination therefore chemosensitizes cancer stem cells of colorectal cancer, which in this case have been isolated from patient. The data in FIG. 4 demonstrate that the synergistic effect is also confirmed in gastric and breast cancer cell lines. In FIG. 5 it is demonstrated how the effect obtained by administering to HT29 and HCT116 colorectal cancer lines the chemotherapeutic compounds for 72 h is comparable, for a fixed dose, to that obtained by treating with a combination of EM127 for 72 h and chemotherapeutic compound for 24 h, thereby being possible to reduce the times of exposure to the chemotherapeutic compound.

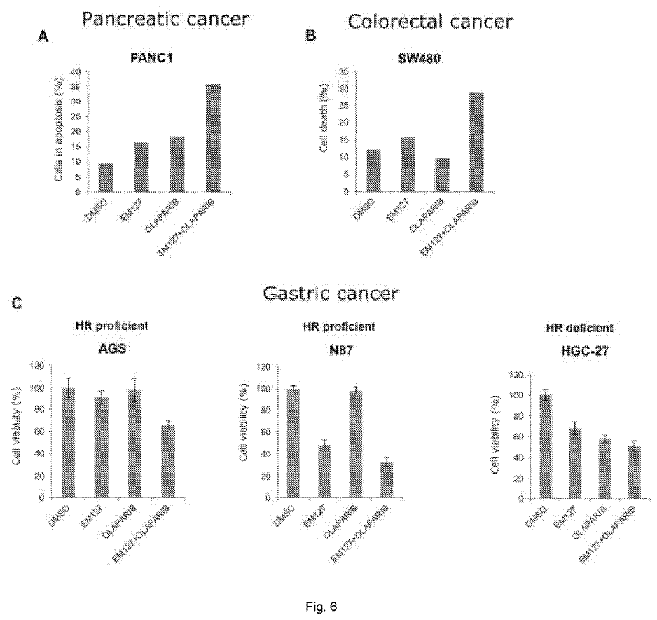

The operational synergy of EM127 with PARP inhibitor Olaparib is demonstrated in FIG. 6, where the combined treatment shows an increase of cells undergoing programmed death and a reduction, therefore, of cell viability, an effect verifiable in cell lines originating from various cancer types. In particular, the effect is verifiable in those cell lines able to repair DNA via homologous recombination (HR-proficient). It has previously been described that those unable to do it (HR-deficient) are sensitive to treatment with Olaparib alone.

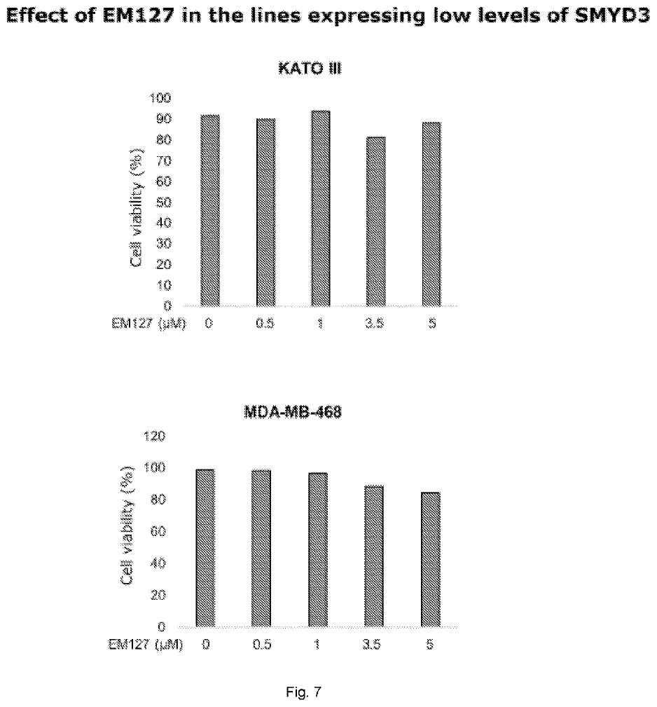

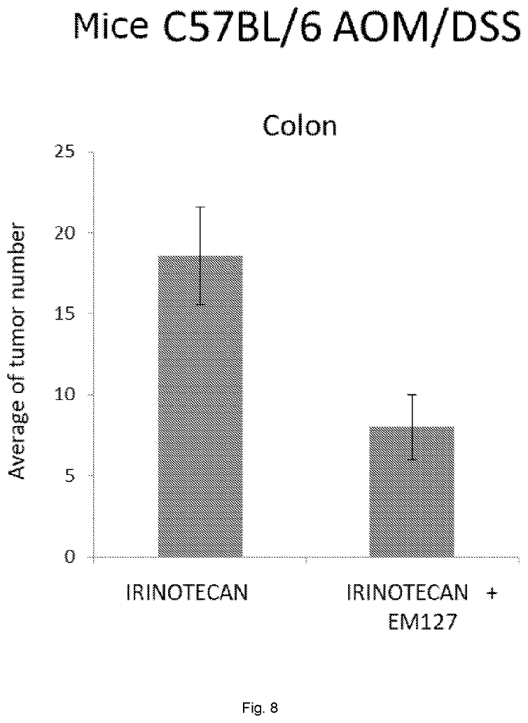

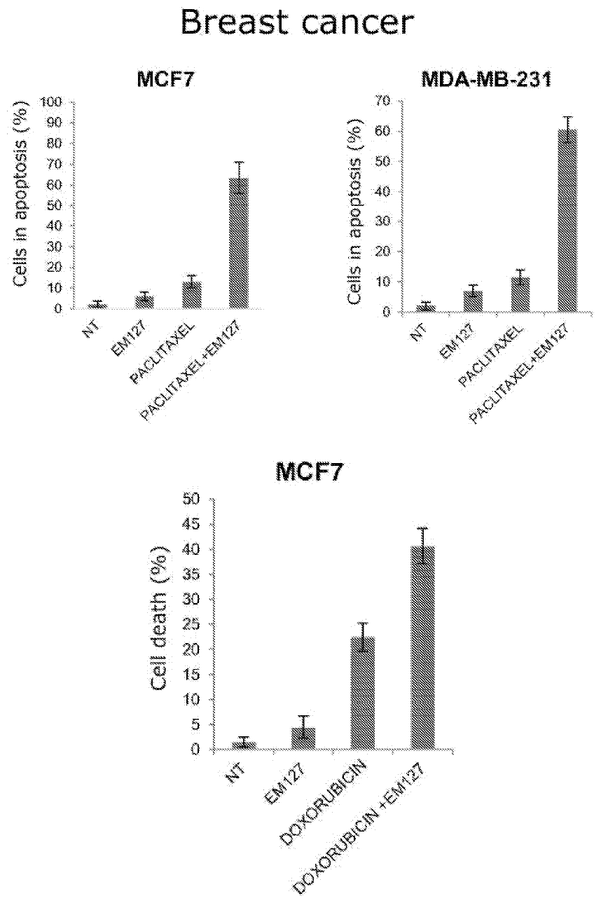

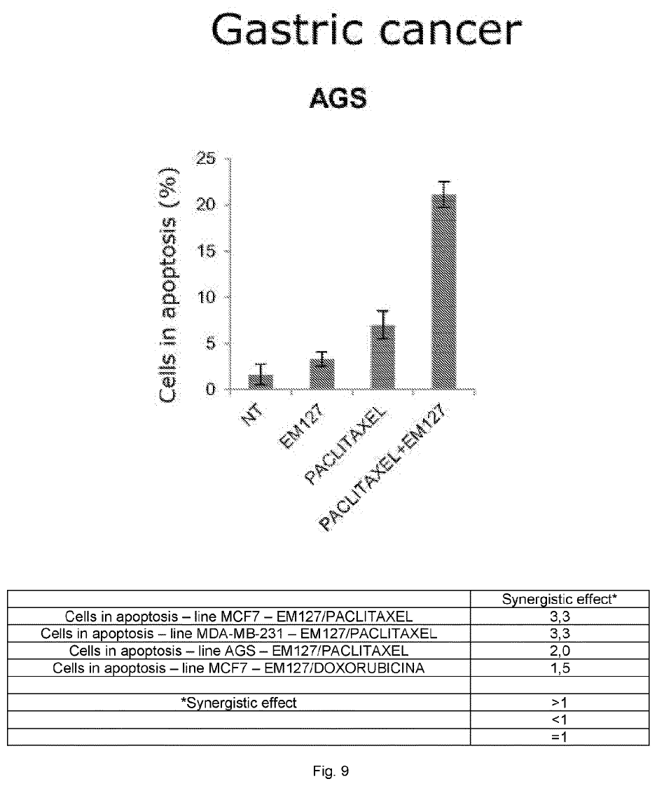

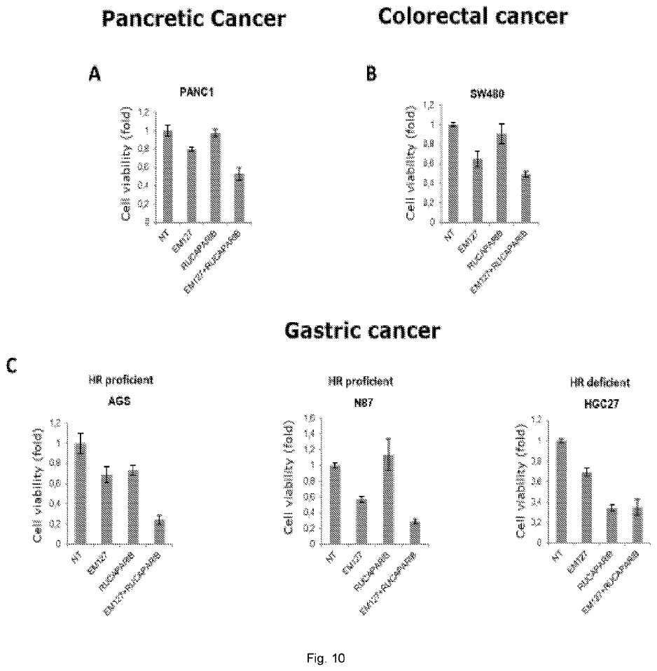

In FIG. 7 it is apparent how EM127 alone has no effect in lines expressing low SMYD3 protein levels, whereas FIG. 8 shows the effect of the combined treatment analyzed on a murine model of colorectal cancer called AOM/DSS, obtained by administration of these two chemical compounds (Azoxymethane+Dextran sulfate sodium), showing that the combination of EM127 with the chemotherapeutic compound Irinotecan reduces the number of tumors present at the level of the colon compared to those found in samples treated with the chemotherapeutic compound alone. Moreover, in FIG. 9 it is reported the assessment of EM127 effect in sensitizing cancer cells to chemotherapeutic compounds Doxorubicin and Paclitaxel, repeated by using breast cancer (MCF7, MDA-MB-231) and gastric cancer (AGS) lines. To this end, an optimization of chemotherapeutic compound concentrations, specific for each cell line, and an assessment of the nonviable/apoptotic cell population were performed. The analysis revealed that the combination of EM127 with Doxorubicin or Paclitaxel has a synergistic effect in all cases, as highlighted by the table. Also in FIG. 10, cell viability data confirm the synergistic effect of EM127 combined with another PARP inhibitor (Rucaparib) in cell lines of gastric cancer N87 and HGC-27 and AGS, pancreatic cancer (PANC1) and colorectal cancer (SW480).

Hence, object of the present invention is:

An association of a compound of formula (I)

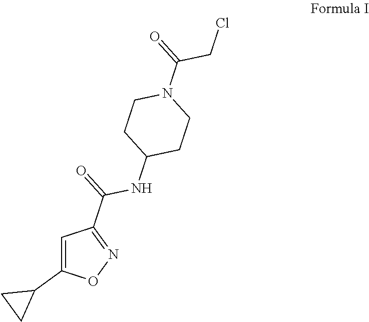 [SMILES: O=C(NC1CCN(C(=O)CCl)CC1)c1cc(C2CC2)on1]

or a pharmaceutically acceptable salt thereof, and one or more chemotherapeutic compounds and/or PARP inhibitors;
a pharmaceutical composition comprising the association, defined by any one of the embodiments described herein, and at least one pharmaceutically acceptable carrier or excipient;
a kit of parts comprising the association, defined by any one of the embodiments described herein, wherein said compound of formula I, said one or more chemotherapeutic compounds and/or said one or more PARP inhibitors are provided in separate aliquots for concomitant or sequential administration;
and their use in the treatment of pathologies in which it is therapeutically advantageous the inhibition of a histone methyltransferase or of a protein of the SMYD family, or of SMYD3. Further aspects and embodiments of the invention will be clarified in the detailed description of the invention.

### GLOSSARY

The term “pharmaceutically active agent” in the present description refers to a compound that when administered at suitable concentrations exhibits a pharmacological activity.

The term “PARP inhibitor” refers to a substance blocking an enzyme, called PARP, in the cell. PARP, also known as poly(ADP-ribose) polymerase inhibitor, is active in the repair of DNA when the latter is damaged. In cancer treatment, PARP blocking can help prevent tumor cells from repairing their damaged DNA, causing their death. PARP inhibitors are a type of targeted therapy.

The term “histone methyltransferase” refers to an enzyme able to transfer a methyl group specifically on a histone tail residue. This methylation modifies histone interaction with the associated proteins and therefore reshapes chromatin structure.

The term “SMYD family” refers to five different proteins (SMYD1, SMYD2, SMYD3, SMYD4 and SMYD5) exhibiting a methyltransferase activity on histone and non-histone substrates.

The term “concomitant administration” refers to the administration of more than at least two compounds in a therapy, which are administered at the same time instead of one after the other.

The term “sequential administration” refers to the administration of at least two compounds in a therapy, which are administered at different times of the therapy.

The term “therapeutically effective amount” refers to an amount that is effective in therapy, or an amount sufficient to provide a desired therapeutic effect. An amount that is effective in therapy is an amount producing one or more desired biological activities in the treatment or prevention of a disease. In the present case, in the treatment of a pathology in which it is therapeutically advantageous the inhibition of a histone methyltransferase or of a protein of the SMYD family, or of SMYD3.

### DETAILED DESCRIPTION OF THE FIGURES

FIG. 1 shows cell viability data (1A) in colorectal cancer cells SW480 and Caco2. The treatment with EM127 for 72 h in combination with a chemotherapeutic compound (Oxaliplatin, 5-Fluorouracil or Irinotecan, 10 μM) administered for the last 24 h, reduces cell viability with a greater effect compared to the treatment with chemotherapeutic compound (1A) alone. For HCT116 cells instead, a dead (apoptotic) cell percentage increased thanks to the combined treatment (1B) is shown.

FIG. 2 shows the synergy obtained thanks to the combined EM127+chemotherapeutic compounds (Oxaliplatin or Irinotecan, 10 μM) treatment observed in 3D cell models obtained from HCT116 cell line (HCT116-derived tumor spheroids) by immunofluorescence analysis of live (first column) and dead (second column) cells.

FIG. 3 shows the synergistic effect of the combined treatment EM127+chemotherapeutic compounds (Oxaliplatin, 5-Fluorouracil or Irinotecan, 10 μM) by analysis of cell viability (3A), cancer stem cell ability to migrate (3B) and to form colonies (3C).

FIG. 4 shows cell viability data confirming the synergistic effect of EM127 combined with chemotherapeutic compounds (Paclitaxel 10 μM, Oxaliplatin 10 μM, Doxorubicin 1 μM) in gastric cancer, N87 and HGC-27, and breast cancer, MDA-MB-231, cell lines.

FIG. 5 shows: on the left, the chemosensitization of HT29 and HCT116 cells obtained by administering EM127 for 72 h in combination with the chemotherapeutic compounds Oxaliplatin, 5-Fluorouracil or Irinotecan, (10 μM) administered in the last 24 h; on the right, the effect of the single treatment with the three different chemotherapeutic compounds for 72 h at increasing concentrations. With the combination of the two compounds, EM127 for 72 h and the chemotherapeutic compound for 24 h, it is obtained a reduction of about 50% of cell viability, obtainable in single-compound treatment for a longer time (72 h).

FIG. 6 shows an increase of cell death (6A, B) and a reduction of cell viability (6C) in cells of pancreatic cancer PANC1, colorectal cancer SW80 and gastric cancer AGS, N87, HGC-27 treated with EM127 and Olaparib (10 μM) for 72 h, comparing the effect of the combined treatment with that of the single compound. In FIG. 6C, the combined treatment shows a reduction of cell viability only for HR-proficient cell lines.

FIG. 7 shows the absence of effect of the treatment with EM127 by viability analysis of KATO III and MDA-MB-468 cancer cells exhibiting low SMYD3 protein levels.

FIG. 8 shows the synergistic effect of the combined treatment EM127+Irinotecan in a murine AOM/DSS model of colorectal cancer. The combination of EM127 with the chemotherapeutic compound Irinotecan reduces the number of tumors present at the level of the colon, compared to those found in samples treated with the sole chemotherapeutic compound.

FIG. 9 shows the synergistic effect of EM127 in sensitizing cancer cells along with chemotherapeutic compounds Doxorubicin and Paclitaxel, repeated by using breast cancer (MCF7, MDA-MB-231) and gastric cancer (AGS) lines. Moreover, the Figure shows the calculation of the synergistic effect which, in all cases, proves to be higher than 1.

FIG. 10 shows cell viability data confirming the synergistic effect of EM127 combined with a second PARP inhibitor (Rucaparib) in cell lines of gastric cancer N87 and HGC-27 and AGS, pancreatic cancer (PANC1) and colorectal cancer (SW480).

### DETAILED DESCRIPTION

The present invention refers to an association of a compound of formula (I)

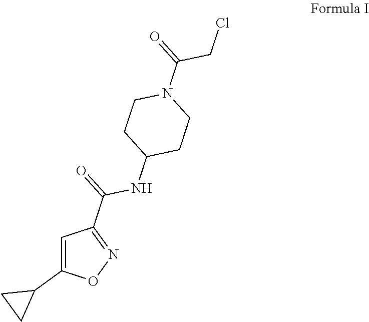 [SMILES: O=C(NC1CCN(C(=O)CCl)CC1)c1cc(C2CC2)on1]

or a pharmaceutically acceptable salt thereof, and one or more chemotherapeutic compounds and/or PARP inhibitors.

In one embodiment, said one or more chemotherapeutic compounds are selected from Irinotecan, Oxaliplatin, 5-Fluorouracil, Doxorubicin, Paclitaxel, or pharmaceutically acceptable salts thereof.

Irinotecan is a chemotherapeutic antineoplastic drug belonging to the class of camptothecins (drugs extracted from the bark of Camptotheca acuminata), having formula:

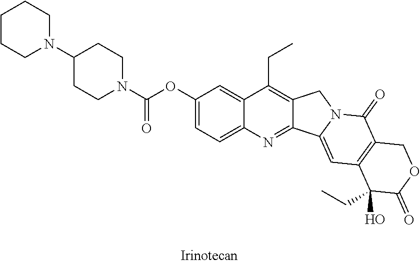

Oxaliplatin is a chemotherapeutic antineoplastic agent able to interfere with all cell cycle phases by binding to DNA through the formation of crosslinks across complementary strands. It is a third-generation platinum analog (diaminocyclohexane derivative), analogous to cisplatin.

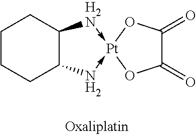

Fluorouracil (5-Fluorouracil, 5-FU, or fluoruracil), a pyrimidine analog, is an anticancer chemotherapeutic agent belonging to the antimetabolite family. Used in clinical oncology since about 40 years ago, it is one of the drugs most used in adjuvant therapy of colorectal and pancreatic tumors. Usually, it is administered in therapeutic protocols combining it with folinic acid (Leucovorin). Fluorouracil is a thymine-analog antimetabolite. How it works is not entirely clear, but it is believed to involve blocking the action of thymidilate synthase and thus stopping DNA synthesis.

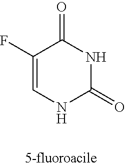

Doxorubicin (also known under the name adriamycin) is an antineoplastic antibiotic of the anthracycline family, having a broad anti-tumor spectrum. The drug binds to cell DNA, inhibiting nucleic acid synthesis and mitosis and causing chromosomal aberrations. Doxorubicin is not phase-specific but is active above all in phase S of the cell cycle. In vitro, the cells most sensitive to Doxorubicin are the cardiac ones, those of sarcomas and melanomas, standard muscle, and skin fibroblasts. As sensitive, though variably, are bone marrow, oral and gastrointestinal mucosa and hair bulbs, i.e. rapidly proliferating cells. Doxorubicin also exhibits antibacterial and immunosuppressive activities.

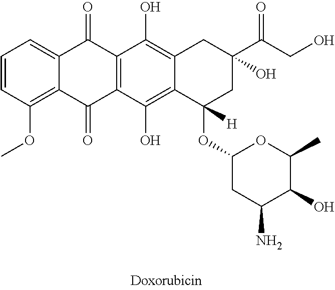

Paclitaxel is a chemotherapeutic agent belonging to the taxane group. It is used for the treatment of various neoplasms, in particular of advanced-stage ovarian carcinoma; breast carcinoma, advanced-stage non-small cell lung carcinoma and AIDS-associated Kaposi's sarcoma.

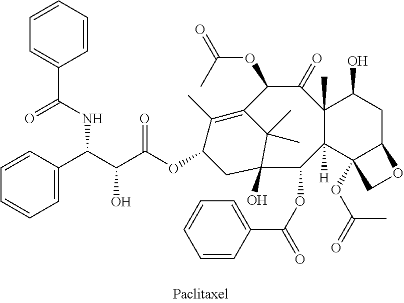

In a preferred embodiment, said chemotherapeutic agent is Irinotecan or a pharmaceutically acceptable salt thereof.

Object of the invention is also the association between the compound of formula I and cocktails of anticancer agents (compounds) comprising at least one of the anticancer agents (compounds) indicated above.

Such as, e.g.:
In one embodiment, said PARP inhibitor compounds are selected from Olaparib, Niraparib and Rucaparib or pharmaceutically acceptable salts thereof.

Olaparib is a compound used for the maintenance treatment of BRCA-mutated advanced ovarian carcinoma in adults. It is a PARP inhibitor, inhibiting poly-ADP ribose polymerase (PARP), an enzyme involved in DNA repair. It acts against cancers in people with hereditary BRCA1 or BRCA2 mutations, which include some ovarian, breast and prostate cancers. Olaparib is indicated for the treatment of breast, ovarian, Fallopian tube, peritoneal, pancreatic, and prostate cancer.

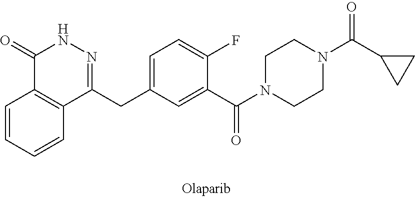

Niraparib is an anticancer agent used for the treatment of epithelial ovarian, fallopian tube, or primary peritoneal cancer.

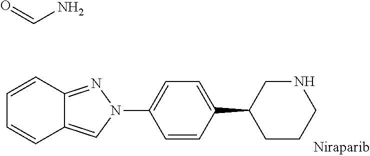

Rucaparib is a PARP inhibitor used as orally administered anticancer agent. Rucaparib is considered as a first-line active ingredient, targeting the DNA repair enzyme poly-ADP ribose polymerase-1 (PARP-1).

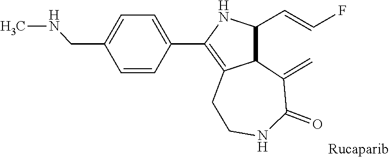

In a preferred embodiment, said PARP inhibitor is Olaparib, or a pharmaceutically acceptable salt thereof.

In one embodiment, the pharmaceutically acceptable salts of said compound of formula I and said pharmaceutically active agents are salts formed with organic acids such as oxalic acid, tartaric acid, maleic acid, succinic acid, or citric acid, or with inorganic acids such as nitric acid, hydrochloric acid, sulfuric acid, and phosphoric acid.

In one embodiment, in the association of the invention said compound of formula I and said one or more chemotherapeutic compounds and/or PARP inhibitors are provided in separate administration forms or in the same administration form.

The association described herein can be formulated as pharmaceutical composition, but also as the kit of parts for concomitant or sequential administration.

According to the invention, the pharmaceutical composition could comprise the association of the compound of formula I

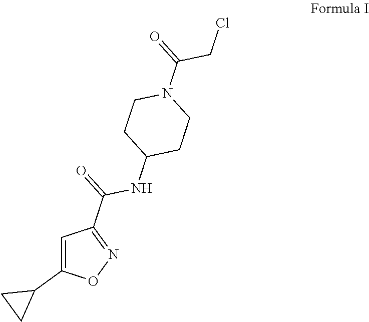 [SMILES: O=C(NC1CCN(C(=O)CCl)CC1)c1cc(C2CC2)on1]

or a pharmaceutically acceptable salt thereof, and a pharmaceutically active agent selected from a group consisting of chemotherapeutic agents or PARP inhibitors as defined above, and at least one pharmacologically acceptable excipient or carrier.

Said carriers and/or excipients, depending on the preferred administration form, are well-known to a person skilled in the art.

In one embodiment the pharmaceutical composition will comprise said compound of formula I and said one or more chemotherapeutic or PARP inhibitors as sole therapeutically active agents, or it could comprise additional pharmacologically active agents as described hereinafter.

Said additional pharmacologically active agents can be selected from anti-inflammatory and/or hypoglycemic agents.

The composition could be prepared by using procedures well-known in the pharmaceutical art. The composition of the present invention can be administered, over a therapy, in a therapeutically or pharmaceutically effective amount.

In one embodiment, said compound of formula I or a pharmaceutically acceptable salt thereof, and said pharmaceutically active agent, are provided in one or more dosage forms at a therapeutically effective concentration.

A non-limiting example of formulation of the pharmaceutical composition or of the aliquots of the kit of parts as described or claimed herein provides formulations suitable for oral, sublingual, nasal, parenteral, topical, transdermal, intravenous, vaginal, or rectal administration.

In one embodiment, the pharmaceutical composition is in form of tablets, suppositories, pills, elastic and hard soft-gelatin capsules, powders, solutions, suspensions, aerosol, and is administered orally, buccally, nasally, parenterally, topically, transdermally, vaginally, or rectally.

In another embodiment, the association of the invention could be formulated as kit of parts, suitable for the concomitant or sequential administration of the various active principles, wherein said one or more chemotherapeutic compounds and/or said one or more PARP inhibitors are provided in separate aliquots.

In this case, therefore, the compound of formula I and the chemotherapeutic compound(s) or PARP inhibitor(s) forming the association can be formulated with suitable carriers and/or excipients, for the concomitant or sequential administration thereof.

Advantageously, the kit could allow different administration routes for the different active principles forming the association described herein.

In this case as well, said compound of formula I or a pharmaceutically acceptable salt thereof, and said pharmaceutically active agent, are provided in one or more dosage forms at a therapeutically effective concentration.

As to the kit of parts, said aliquots of said one or more chemotherapeutic compounds and/or said aliquots of said one or more PARP inhibitors are formulated for oral, sublingual, nasal, parenteral, topical, transdermal, intravenous, vaginal, or rectal administration.

Given the surprising results obtained by the Inventors (see examples and figures), the association, the pharmaceutical composition or the kit of parts as described and claimed herein are advantageously useful for the treatment of all those pathologies wherein it is therapeutically advantageous for the patient the inhibition of a histone methyltransferase or a protein of the SMYD family, or of SMYD3 and particularly for the treatment of cancerous pathologies, tumor pathologies, musculoskeletal pathologies, inflammatory pathologies, autoimmune pathologies, age-dependent vascular pathologies, and rheumatic pathologies. In particular, pathologies associated to SMYD3 overexpression well-known in the literature. A non-limiting example of said pathologies is reported in Table 2 of Bernard et al “SMYD3: a regulator of epigenetic and signaling pathways in cancer” Clinical Epigenetics 2021 pp 1-19, and comprises pancreatic tumor, breast cancer, colorectal cancer, gastric carcinoma, hepatocellular carcinoma, cervix cancer, prostate tumor, lung cancer, non-small cell lung cancer, ovarian cancer, esophageal cancer, bladder cancer, malignant glioma. Moreover, in Di Yang et al “Histone methyltransferase Smyd3 is a new regulator for vascular senescence” Aging Cell, 19: e13212, SMYD3 role in age-dependent vascular pathologies, which comprise cardiovascular diseases, among which hypertension, diabetes, arteriosclerosis, and insulin resistance syndrome, is reported.

Furthermore, the pathologies associated with SMYD3 overexpression comprise vascular remodeling following an accident, in which SMYD3 role is reported in Yang et al “H3K4 Methyltransferase Smyd3 Mediates Vascular Smooth Muscle Cell Proliferation, Migration, and Neointima Formation” Arteriosclerosis, Thrombosis, and Vascular Biology. 2021; 41:1901-1914, wherein it is shown that SMYD3 promotes vascular smooth cell proliferation during an accident-induced vascular remodeling with the entailed risk of hyperplasia, skeletal muscle atrophy, in which SMYD3 role is made explicit in Proserpio et al. “The methyltransferase SMYD3 mediates the recruitment of transcriptional cofactors at the myostatin and c-Met genes and regulates skeletal muscle atrophy” Genes & Dev. 2013. 27:1299-1312, wherein it is reported that SMYD3 lowering prevents muscle loss and fiber size decrease.

In compliance with Art. 170bis, Paragraph 2, C.P.I (Italian Code of Industrial Property) and in accordance with Art. 21, Paragraph 2 of the Implementing regulation of C.P.I. adopted with Ministerial Decree D.M. 13.1.2010 n.33, it is hereby certified that: The material of animal/plant origin at the base of the invention object of the above-mentioned Application consists of commercially available cell lines (ATCC)
HT29, HCT116, SW480, CACO2 (human colorectal cancer cell lines)
N87, KATO III, HGC-27, AGS (human gastric cancer cell lines)
MDA-MB-231, MDA-MB-468, (human breast cancer cell lines)
PANC1 (human pancreatic cancer cell lines).

In compliance with Art 170bis, Paragraph 3 C.P.I., it is hereby certified that:
The colorectal cancer stem cells used at the base of the invention object of the present application have been isolated from patients, and the aforementioned cells were obtained following the free and informed consent to their collection and use expressed by the person from whom said material has been collected, under the regulations in force; and
C57BL/6 mice for the AOM/DSS murine model of colorectal cancer were not genetically modified.

Throughout the description, the term “comprising” can be replaced by the term “consisting of”.

### Examples

### Cell Cultures and Murine Models

The human cell lines HT29, HCT116, SW480, MDA-MB-231, MDA-MB-468, HGC-27, and PANC1 were cultured in DMEM culture medium, supplemented with 10% fetal bovine serum (FBS); N87, AGS and KATO III were cultured in RPMI culture medium supplemented with 10% FBS; CACO2 were cultured in DMEM culture medium, supplemented with 20% FBS and nonessential amino acids. All cell lines were cultured in the presence of 1% antibiotics (Penicillin and Streptomycin), at 37 °C in an atmosphere of 5% CO₂ and always maintained in a subconfluence state.

The tumorspheres deriving from the continuous cell line HCT116 “HCT116-derived tumor spheroids” were obtained by using a suitable DMEM/F12 Advanced culture medium supplemented with 1× Glutamine, 1× Penicillin/streptomycin, 0.6% glucose, 1×B27, 1×N2, 20 ng/ml EGF, 10 ng/ml bFGF, and cultured in media surface-treated to ensure a very low cell adhesion.

For the study, the murine model of chemically induced colitis-associated carcinogenesis (CAC) was used. C57BL/6 mice were treated intraperitoneally with 12 mg/kg azoxymethane (AOM). Dextran sulfate sodium (DSS) was then administered, at 2% in water for five days, followed by two weeks with water only. This cycle was repeated thrice. At +10 days from the last DSS administration, the Inventors proceeded with the treatment with 20 mg/kg EM127 and/or 20 mg/kg Irinotecan, or with the carrier alone, intraperitoneally, for a total of 14 days.

### Cytotoxicity Assays

Cell viability analysis was performed by using WST-1 kit (Roche), allowing to measure viable cell metabolic activity. In short, the quantitation of mitochondrial dehydrogenase activity is assessed by soluble tetrazolium salt (MTT, colorless) conversion into its corresponding product, formazane (colored). The quantitative assessment of salt conversion into its colored form is then performed by spectrophotometrically calculating absorption at 450 nm. Substrate conversion levels are therefore proportional to enzymatic activity which, in turn, represents a marker of cell viability and proliferation.

Analysis of non-viable cell percentage was carried out by counting live cells and dead cells after staining with 0.01% Trypan Blue, a dye permeating cells only in the condition in which plasma membrane integrity is compromised. In short, after collecting the culture medium and the plate-adhering cells, detached by enzymatic treatment with 1% trypsin/EDTA, the pellet obtained by centrifuging was resuspended in an adequate volume of DPBS and 10 μl of cell suspension were diluted in an equal volume of Trypan blue. The counting was performed with a phase-contrast optical microscope, using a Burker chamber.

Analysis of the percentage of cells undergoing programmed death, referred to as apoptosis, was performed by Annexin V assay. The concentrations of live cells, cells in early apoptosis, in total apoptosis and dead cells were measured with Muse Annexin V and Dead Cell Reagent kit (Luminex). 5×10⁴ cells/mL were stained with the reagent provided by the kit. After 20 min incubation, fluorescence is read with Guava® Muse® Cell Analyzer. The test was performed on the following cell lines: HCT116, PANC1, SW480, MDA-MB-231, MCF7, AGS.

To assess cell viability on HCT116-derived tumorspheres, LIVE/DEAD® Viability/Cytotoxicity Assay kit (Thermo Fisher) was used, allowing to discriminate live cells from dead ones with two probes measuring intracellular enzymatic activity and plasma membrane integrity. Tumorspheres were incubated with the reagent provided by the kit and then visualized with a fluorescence microscope. Live cells are stained with reagent calcein and generate a green fluorescence signal; dead cells are stained with ethidium homodimer, generating a red signal.

The synergistic effect as shown in FIG. 9 was calculated as follows:

$$Synergistic effect = \frac{( \% of cells with combined treatment )}{\begin{matrix}( \% cells with EM 127 treatment ) + \\ ( \% cells with CHT / PARPi treatment )\end{matrix}}$$

From the above formula it is inferred that >1 values have demonstrated synergistic effects, <1 values have inverted synergistic effects and values near to 1 demonstrate no synergy.

### Clonogenic Assay

For the clonogenic assay, previously dissociated colorectal cancer tumorspheres were plated in triplicate using 500 cells per experimental point and resuspended in 0.3% agarose on a 0.5% agarose layer. Subsequently, they were treated with EM127 and chemotherapeutic compounds.

### Migration Assay

To assess cell motility, colorectal cancer tumorspheres were suspended in 200 μl non-supplemented stem cell culture medium and plated in the top section of a Boyden culture chamber, which is separated from the bottom part by a polycarbonate membrane allowing cells to migrate. The underlying section contains 600 μl of stem cell medium supplemented with 20 ng/ml EGF and 10 ng/ml basic FGF and chemotherapeutic compounds.

1. A pharmaceutical composition comprising a compound of formula (I) 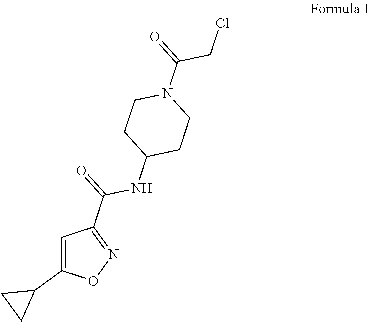 [SMILES: O=C(NC1CCN(C(=O)CCl)CC1)c1cc(C2CC2)on1]
or a pharmaceutically acceptable salt thereof, and one or more chemotherapeutic compounds and/or PARP inhibitors.

2. The pharmaceutical composition of claim 1, wherein said one or more chemotherapeutic compounds are selected from Irinotecan, Oxaliplatin, 5-Fluorouracil, Doxorubicin, Paclitaxel, and pharmaceutically acceptable salts thereof.

3. The pharmaceutical composition of claim 1, wherein said one or more PARP inhibitor compounds are selected from Olaparib, Niraparib, Rucaparib, and pharmaceutically acceptable salts thereof.

4. The pharmaceutical composition of claim 1, further comprising one or more anti-inflammatory and/or hypoglycemic compounds.

5. (canceled)

6. The pharmaceutical composition of claim 1, further comprising at least one pharmaceutically acceptable carrier or excipient.

7. The pharmaceutical composition of claim 6, wherein said composition is formulated for oral, sublingual, nasal, parenteral, topical, transdermal, intravenous, vaginal, or rectal administration.

8. A kit of parts comprising a compound of formula (I) 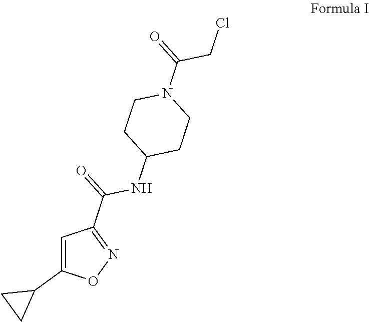 [SMILES: O=C(NC1CCN(C(=O)CCl)CC1)c1cc(C2CC2)on1]
or a pharmaceutically acceptable salt thereof, and one or more chemotherapeutic compounds and/or PARP inhibitors, wherein said compound of formula I, said one or more chemotherapeutic compounds and/or said one or more PARP inhibitors are provided in separate aliquots for concomitant or sequential administration.

9. The kit of parts of claim 8, wherein said aliquots of said compound of formula I, said aliquots of said one or more chemotherapeutic compounds and/or said aliquots of said one or more PARP inhibitors are formulated for oral, sublingual, nasal, parenteral, topical, transdermal, intravenous, vaginal, or rectal administration.

10. The kit of parts of claim 9, wherein said aliquots of said compound of formula I and said aliquots of one or more chemotherapeutic compounds and/or said aliquots of one or more PARP inhibitors are formulated for different types of administration from each other.

11. A method of treating a pathology for which inhibition of a histone methyltransferase or a protein of the SMYD family, or of SMYD3, is advantageous, the method comprising administering therapeutically effective amounts of a compound of formula (I) 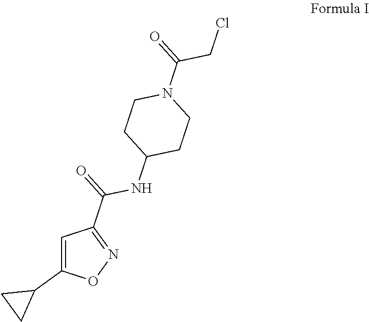 [SMILES: O=C(NC1CCN(C(=O)CCl)CC1)c1cc(C2CC2)on1]
or a pharmaceutically acceptable salt thereof, and one or more chemotherapeutic compounds and/or PARP inhibitors.

12. The method of claim 11, wherein said pathology is selected from a cancerous pathology, a tumor pathology, a musculoskeletal pathology, an inflammatory pathology, an autoimmune pathology, a cardiovascular pathology, and a rheumatic pathology.

13. The method of claim 11, wherein said pathology is selected from pancreatic tumor, mammary tumor, breast cancer, colorectal cancer, gastric carcinoma, hepatocellular carcinoma, cervix cancer, prostate tumor, lung cancer, non-small cell lung cancer, ovarian cancer, esophageal cancer, bladder cancer, and malignant glioma.

14. The method of claim 11, wherein said one or more chemotherapeutic compounds are selected from Irinotecan, Oxaliplatin, 5-Fluorouracil, Doxorubicin, Paclitaxel, and pharmaceutically acceptable salts thereof.

15. The method of claim 11, wherein said one or more PARP inhibitor compounds are selected from Olaparib, Niraparib, Rucaparib, and pharmaceutically acceptable salts thereof.

16. The method of claim 11, further comprising one or more anti-inflammatory and/or hypoglycemic compounds.

17. The method of claim 11, wherein said compound of formula I and said one or more chemotherapeutic compounds and/or PARP inhibitors are administered in separate administration forms.

18. The method of claim 11, wherein said compound of formula I and said one or more chemotherapeutic compounds and/or PARP inhibitors are administered in the same administration form.

19. The method of claim 11, wherein the one or more chemotherapeutic compounds and/or PARP inhibitors are selected from Irinotecan, Oxaliplatin, 5-Fluorouracil, Doxorubicin, Paclitaxel, Olaparib, Rucaparib, and pharmaceutically acceptable salts thereof.

20. The method of claim 19, wherein said pathology is selected from pancreatic cancer, breast cancer, colorectal cancer, and gastric cancer.
